SEQUENCE LISTING (12 sequences)

SEQ ID NO: 1 - DNA, 22 bp, human
gctcccggctcgcctccttacc

SEQ ID NO: 2 - DNA, 24 bp, Human
cctggccctgctagtggggaactc

SEQ ID NO: 3 - DNA, 25 bp, Human
aggtcgcttgaggcgaaatccttcc

SEQ ID NO: 4 - DNA, 30 bp, Human
tcccacacccaccctcttctacccttctga

SEQ ID NO: 5 - DNA, 24 bp, Human
tattgaaaggggccccagaaggtc

SEQ ID NO: 6 - DNA, 94 bp, HUMAN
tgtgtgtgtgtgtgtgagagagagagtgtgtgtgtgtgagaatttgtgtgtgtgtgagtgtgtgtgtgagtgtgtgtgtgagtgtgtgtgtgtg

SEQ ID NO: 7 - DNA, 88 bp, HUMAN
tgtgtgtgtgtgtgtgagagagtgtgtgtgtgtgagaatttgtgtgtgtgtgagtgtgtgtgagtgtgtgtgtgagtgtgtgtgtgtg

SEQ ID NO: 8 - DNA, 76 bp, HUMAN
tgtgtgtgtgtgtgtgtgagagagtgtgtgtgtgagaatttgtgtgtgtgtgagtgtgtgtgtgagtgtgtgtgtg

SEQ ID NO: 9 - DNA, 90 bp, HUMAN
tgtgtgtgtgtgtgtgtgagagagagtgtgtgtgtgtgagaatttgtgtgtgtgtgagtgtgtgtgtgagtgtgtgtgtgtgtgtgtgtg

SEQ ID NO: 10 - DNA, 88 bp, HUMAN
tgtgtgtgtgtgtgtgagagagagtgtgtgtgtgtgagagagagtgtgtgtgtgagtgtgtgtgtgagtgtgtgtgtgtgtgtgtgtg

SEQ ID NO: 11 - DNA, 88 bp, HUMAN
tgtgtgtgtgtgtgtgtgagagagagtgtgtgtgtgtgagaatttgtgtgtgtgtgagtgtgtgtgtgagtgtgtgtgtgtgtgtgtg

SEQ ID NO: 12 - DNA, 86 bp, HUMAN
tgtgtgtgtgtgtgtgtgagagagtgtgtgtgtgagaatttgtgtgtgtgtgagtgtgtgtgtgagtgtgtgtgtgtgtgtgtgtg

(19) United States
(12) Patent Application Publication
(10) Pub. No.: US 2006/0234274 A1
(43) Pub. Date: Oct. 19, 2006

(54) Methods of diagnosis

(75) Inventors: Andrew Neil Shelling, Auckland (NZ); Sarah Elizabeth Harris, Fife (GB)

Correspondence Address: NIXON & VANDERHYE, PC, 901 NORTH GLEBE ROAD, 11TH FLOOR, ARLINGTON, VA, 22203, US

(73) Assignee: Auckland UniServices Limited, Auckland (NZ)

(21) Appl. No.: 11/391,736

(22) Filed: Mar. 29, 2006

(30) Foreign Application Priority Data
Mar. 29, 2005 (AU) 2005901513

(51) Int. Cl. C12Q 1/68

(52) U.S. Cl. 435/6

(57) ABSTRACT

A method to detect whether a female subject is predisposed to POF, the method comprising analysing at least one or more polymorphism in the INHA gene chosen from the group consisting: −124A>G; −16C>T; TG repeat (as herein after described); and one or more polymorphism in the INHA gene which is in linkage disequilibrium with one or more of −124A>G, −16C>T or TG repeat (as herein after described).

### FIELD

The present invention relates to methods of diagnosis, particularly those of, use in determining a female subject's susceptibility or predisposition to premature ovarian failure (POF), diagnosing POF or predicting onset of infertility.

### BACKGROUND

Premature ovarian failure (POF) is a syndrome causing secondary amenorrhea, hypoestrogenism, elevated gonadotrophins, and ultimately leads to the cessation of ovarian function under the age of 40 years. It affects 1% of all women and occurs in 0.1% before the age of 30 years (11). POF may result from either a decreased number of follicles being formed during ovarian development, or by an increased rate of follicle loss. Alternatively, follicles may be present, but unresponsive to hormonal stimulation. The most commonly known causes of POF are X-chromosome abnormalities (12), but in the majority of women, with a normal karyotype, there is no known cause. However, many women with POF have other female family members who also have POF, suggesting an inherited predisposition to the condition.

The most immediate concern for women with POF is the menopausal symptoms they experience due to the decrease in circulating oestradiol coupled with the psychological implications of these symptoms. The menopausal symptoms include hot flushes, night sweats, insomnia, palpitations, headaches, incontinence, and dyspareunia as a result of vaginal dryness. The psychological implications of POF include those associated with menopause such as forgetfulness, poor concentration, irritability and mood swings.

A second consequence of POF is the loss of fertility. Even though some women will spontaneously ovulate and achieve a natural pregnancy most women with POF will not. Infertility treatment is difficult, as in many cases the ovary does not have any follicles left. In the cases where follicles can be detected by biopsy, the ovary has become unresponsive to FSH. Therefore, most women with POF can either choose to adopt children or undergo donor egg IVF. However, obtaining donor eggs can be difficult and the procedure can be very expensive.

The long-term consequences of POF are caused by the increased length of time the body will be without ovarian oestrogen. The risk of osteoporosis and cardiovascular diseases increases after menopause due to a decrease in oestrogen that appears to provide a protective effect against these diseases. Women with POF have reduced oestrogen levels for between 20 to 30 years longer than normal women. Therefore, the risk of these diseases is thought to be much greater in women with POF. For this reason patients with POF are prescribed hormone replacement therapy (HRT). However, these women have an additional concern. The prolonged use of HRT has been associated with an increase in the risk of acquiring breast cancer, endometrial cancer and gallstones. Until research into the long-term effects of HRT in women with POF has been conducted this issue will still be a major concern for these women.

Recent research in relation to POF has identified for the first time that alterations in genes encoding subunits of the protein Inhibin may be indicative of POF, or at least a predisposition thereto (WO 00/50638).

Inhibins are dimeric glycoproteins consisting of a common inhibin alpha subunit (INHA, 14 kDa) covalently linked to one of two related inhibin beta subunits (INHBA, 18 kDa and INHBB, 18 kDa), which respectively form inhibin A and inhibin B. Inhibins were originally isolated from porcine and bovine follicular fluid (1-4) and the main production sites are now known to be the granulosa cells in the female and sertoli cells in the male (5). Inhibin A and B both inhibit the secretion of FSH, which is involved in the recruitment and development of ovarian follicles during folliculogenesis, but are themselves secreted by the ovarian follicles at different times during the menstrual cycle. Inhibin B is secreted during the follicular phase whereas inhibin A is restricted to the luteal phase (6, 7). The activins, which stimulate FSH activity, are composed of homo- or heterodimers of the inhibin beta subunits (8, 9). The inhibin subunits are all members of the TGF-β superfamily, a group of molecules that are involved in cell growth and differentiation.

Details of the publications referred to herein are collected at the end of the description.

### OBJECT

It is an object of the present invention to provide a method which is of use in detecting whether a female subject is predisposed to premature ovarian failure (POF), in predicting onset of infertility, and/or in diagnosing POF.

### STATEMENT OF INVENTION

In accordance with the present invention the inventors have identified that certain polymorphisms within the nucleic acid region up-stream of the translational start site of the INHA gene may provide valuable information about a female subject's predisposition or susceptibility to POF, and which may help in diagnosis of POF or in predicting onset of infertility.

In one aspect of the present invention there is provided a method to detect whether a female subject is predisposed to POF, the method comprising analysing at least one or more polymorphism in the INHA gene chosen from the group consisting:

−124A>G;

−16C>T;

TG repeat (as herein after described); and,

One or more polymorphism in the INHA gene which is in linkage disequilibrium with one ore more of −124A>G, −16C>T or TG repeat (as herein after described).

In another aspect, the invention provides a method for diagnosing POF in a female subject, the method comprising analysing at least one or more polymorphism in the INHA gene chosen from the group consisting:

−124A>G;

−16C>T;

TG repeat (as herein after described); and,

One or more polymorphism in the INHA gene which is in linkage disequilibrium with one ore more of −124A>G, −16C>T or TG repeat (as herein after described).

In another aspect the invention provides a method for predicting the onset of infertility in a female subject, the method comprising at least the analysis of one or more polymorphism in the INHA gene chosen from the group consisting:

−124A>G;

−16C>T;

TG repeat (as herein after described); and

One or more polymorphism in the INHA gene which is in linkage disequilibrium with one ore more of −124A>G, −16C>T or TG repeat (as herein after described).

Preferably, the one or more polymorphism in linkage disequilibrium is 531C>T.

In yet another aspect of the present invention there is provided a method to detect whether a female subject is predisposed to POF, the method comprising determining the subjects genotype in respect of a TG repeat polymorphism (as herein after described) in the INHA gene by analysing one or more of the polymorphisms chosen from the group consisting:

−16C>T; and,

−124A>G.

In another aspect of the invention there is provided a method for diagnosing POF in a female subject, the method comprising determining the subjects genotype in respect of a TG repeat polymorphism (as herein after described) in the INHA gene by analysing one or more of the polymorphisms chosen from the group consisting:

−16C>T; and,

−124A>G.

In another aspect the invention provides a method for predicting the onset of infertility in a female subject, the method comprising determining the subjects genotype in respect of a TG repeat polymorphism (as herein after described) in the INHA gene by analysing one or more of the polymorphisms chosen from the group consisting:

−16C>T; and,

−124A>G.

Heterozygosity or homozygosity for haplotype C (as herein after described) of the TG repeat is indicative of protection against POF.

Heterozygosity or homozygosity for −16T (the T allele) is indicative of protection against POF. Homozygosity for −16C is indicative of susceptibility or predisposition to POF, and potential onset of infertility.

Homozygosity for −124A is indicative of susceptibility or predisposition to POF, and potential onset of infertility. Homozygosity or heterozygosity for −124G is indicative of protection against POF.

Preferably, analysis of one or more polymorphisms occurs via analysis of DNA encoding INHA. Where a genetic marker is present in a coding region of the INHA gene analysis of one or more polymorphisms may occur via analysis of RNA encoding INHA.

Preferably, analysis of one or more of the above mentioned polymorphisms occurs using one or more of:

polymerase chain reaction (PCR);

gel electrophoresis;

Southern blotting;

Nucleic acid sequencing;

restriction fragment length polymorphism (RFLP);

single-strand confirmation polymphism (SSCP);

LCR (ligase chain reaction);

denaturing gradient gel electrophoresis (DGGE);

allele-specific oligonucleotides (ASOs);

proteins which recognize nucleic acid mismatches;

RNAse protection;

oligo array hybridisation;

denaturing HPLC (dHPLC); and,

matrix-assisted laser desorption/ionisation time-of-flight mass spectroscopy (MALDI-TOF MS).

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1: Is a schematic representation of INHA promoter showing the locations of primers used for sequencing (INHA5′F and INHA3′R) and SSCP (INHA3′F and INHA3′R). The major transcription start site and the translation start are also indicated and all numbering is based on the translation start site, beginning at +1. The TG repeat region is shaded and the locations of the −252C>A, −124A>G and −16C>T SNPs are given.

FIG. 2: Illustrates the results of FRFLP/SSCP analysis of INHA promoter. A) FRFLP analysis of −124A>G using Sau3A I. Undigested DNA and digested DNA samples, homozygous for −124G, give a band of 196 bp. DNA samples heterozygous for −124G>A, yield two bands of 196 bp and 168 bp when digested with Sau3A I and samples homozygous for −124A a single band of 168 bp. B) SSCP analysis of the TG repeat and −124A>G. An example of each genotype identified in the New Zealand patients and controls is illustrated.

### PREFERRED EMBODIMENTS(S)

The following is a description of the present invention, including preferred embodiments thereof, given in general terms. The invention is further elucidated from the disclosure given under the section “Examples” which provides experimental data supporting the invention and specific examples thereof.

Recent research in relation to POF has identified for the first time that alterations in genes encoding subunits of the protein Inhibin may be indicative of POF, or at least a predisposition thereto (WO 00/50638). The inventors have now identified the presence of particular alterations (or polymorphisms) in the gene (INHA) encoding the Inhibin alpha subunit which have not been previously described. These alterations show particularly surprising efficacy over those alterations previously described in detecting a female subject's predisposition or susceptibility to developing POF. The inventors believe the alterations described herein may also be of use in methods for diagnosing POF and for predicting the onset of infertility in a subject. The inventor(s) contemplate the particular alterations described herein being of relevance to a greater number of women. Such information may allow female subjects to make appropriate lifestyle choices and may also allow for appropriate therapeutic intervention steps to be taken.

It should be appreciated that “infertility” as used herein encompasses “sub-fertility” where a subject may still have a chance of conceiving but such chance is reduced.

The terms “predisposition” and “susceptibility” as used herein refer to a subject's vulnerability to, or potential risk of, developing POF; in that the nature of a particular genetic marker of the invention may increase a subject's risk of developing POF, while the nature of other genetic markers may decrease the risk of develoment of POF. The terms may be used interchangably herein.

The terms “genetic marker(s)” and “marker(s)” are used herein to generically refer to any one or more (as the context requires) of the polymorphisms of use in the invention.

The human INHA gene nucleic acid sequence and the Inhibin alpha subunit amino acid sequence can be found on GenBank. Accession numbers for INHA mRNA include: M13981 and NM_{—}002191. Accession numbers for the amino acid sequence include: AAA59166 and NP_{—}002182. The BAC clone genomic sequence that was used to design promoter primers (as discussed herein after) has the accession number AC009955.

It should be appreciated that knowledge of the precise positioning of the polymorphisms described herein is important in regard to practising the invention. The established nomenclature system regarding positioning polymorphisms and mutations is outlined in Antonarakis (39 and 40).

The first alteration identified by the inventors is referred to herein as the INHA −16C>T transition. The inventor(s) have identified from a sample of 70 POF patients and 70 controls screened for the presence of the INHA −16C>T transition, that 44.3% of controls carried the T allele compared to only 25.7% of POF patients. This result indicates that the T allele is significantly under-represented in the POF patient population (Fisher's exact test, 2-Tail: P=0.033).

The inventors have also identified in the INHA promoter of 50 POF patients and 50 controls a highly polymorphic imperfect TG repeat. In total, 7 repeat polymorphism haplotypes were typed (A-D and D(i-iii), Table I herein after). The inventors note that haplotype C (the shortest repeat) is indicative of protection against POF.

In addition to the above, the inventors have identified that the −16T allele is linked to the shortest repeat haplotype (haplotype C).

The inventors have also identified a −124A>G SNP which they have shown to be inherited co-ordinately with the polymorphic repeat, with haplotypes A and B having −124A and haplotypes C, D and D(i), −124G. If a subject is homozygous for −124A then they do not carry the protective haplotype C (as haplotype C contains −124G).

On the basis of their findings the inventors believe that the polymorphic repeat, and the SNPs −16C>T, and −124A>G may be used as markers to help determine a female subject's predisposition to POF, and to aid in the diagnosis of POF and in predicting onset of infertility.

The inventor's have further identified a 531C>T SNP which is in linkage disequilibrium with the −16C>T SNP and accordingly may find use in the invention. 531C is associated with −16C and 531T associated with −16T.

It should be appreciated that other SNPs or alternative alterations or polymorphisms in linkage disequilibrium with the polymorphic repeat, and/or the SNPs −16C>T and −124A>G, may be of use in the present invention. Such SNPs could be studied in lieu of those polymorphisms specifically mentioned herein while providing the necessary diagnostic information in accordance with the invention.

The markers may be studied individually, or in combinations of two or more. It should be appreciated that the SNPs specified herein may be used to determine the polymorphic repeat genotype of a subject.

In summary, in respect of the polymorphic repeat, homozygosity or heterozygosity in respect of the shortest repeat (the C haplotype) is indicative of protection against POF. In respect of the SNP −16C>T, homozygosity or heterozygosity for −16T (the T allele) indicates that a subject carries the protective haplotype C (polymorphic repeat). Homozygousity for −16C indicates that a subject does not carry the protective haplotype C and may be susceptible to POF. Regarding the −124A>G SNP, homozygosity for −124A indicates that a subject does not carry the protective haplotype C (polymorphic repeat) and accordingly may be susceptible to POF. Homozygousity or heterozygousity for −124G is indicative of carrying the protective haplotype C.

As used herein phrases such as “protection against POF” and the like should be taken broadly. They are not intended to mean that a subject has absolute protection against developing POF. Such phrases are intended to encompass at least a level of protection against POF or at least a reduced risk of developing POF.

Methods of the invention may rely on the analysis of a single polymorphism as above mentioned, or any combination of two or more of the identified polymorphisms. Further, as the inventors have noted that −16T is associated with the C haplotype of the polymorphic repeat, −124A is associated with haplotypes A and B, and −124G is associated with haplotypes C, D and D(i), these SNPs may be analysed to provide at least an indication of the polymorphic repeat haplotype of a subject without the need for analysing this directly. The inventors believe this is particularly the case for the −16C>T SNP.

Methods of the invention involve the step of taking of a sample from a female subject. Such sample preferably includes one or more cells taken from the subject, including any tissue sample or body fluid. In a preferred embodiment, a blood sample is taken; this may include the use of a Guthries card (blood spot taken at birth). Alternatively, a buccal smear or mouthwash sample may be used. Skilled persons will readily appreciate techniques which may be used to take samples.

A female subject is preferably human. Alternatively, the female subject is another species of animal, for example domestic or companion animals (cats and dogs for example) or livestock (cattle, sheep, horses and the like).

In a preferred embodiment of the invention, following the taking of a sample, the sample is processed to isolate nucleic acid to be analysed in respect of the genetic markers described herein. Generally, genomic DNA will be isolated for further analysis. However, it will be appreciated that in the case of analysis of −16C>T, and in the analysis of 531C>T, mRNA may be isolated for further analysis. In such a case, the mRNA may be converted to cDNA using reverse transcription techniques known in the art.

Techniques for isolating nucleic acids from samples as above mentioned will be readily appreciated by skilled persons. An example of a means for isolating genomic DNA from blood is described herein after under the section entitled “Examples”, with reference to Shelling et al, 2000 (10).

In an alternative form of the invention, it is not necessary to isolate nucleic acid from the sample. For example, in situ analysis of the nucleic acid may occur. This may be done using PCR for example. Skilled persons will readily appreciate appropriate techniques and methodology to this end (see for example, Sambrook and Russell, Molecular Cloning: A Laboratory Manual, 2001, Cold Spring Harbor Laboratory Press, Cold Spring Harbor, N.Y.).

In order to facilitate analysis of the polymorphisms of the invention, nucleic acids may be amplified in a specific manner, using for example PCR or cloning techniques.

In relation to PCR, primers adapted to hybridise to specific regions of the INHA gene, or regions flanking the gene, may be used to specifically amplify the INHA gene, particularly regulatory regions of the gene upstream from the start codon within which the polymorphisms of the invention map. Primers adapted to achieve this result are described herein after under the section entitled “Examples”. However, skilled persons will readily be able to design alternative primers of use to this end having regard to the sequence of INHA and the principles of nucleic acid hybridisation as are well known in the art. Similarly, those primers specifically exemplified herein may be modified while still maintaining their specificity and ability to prime extension during amplification. Such modifications include altering the length of the primers, incorporating non-complementary nucleotide fragments attached to the 5′ end of the primers and altering their sequence so that one or more non-complementary nucleotides are present at a particular position.

A number of available computer programs may be used to assist in primer design; one example is the PrimerSelect program of DNAStar Inc (Madison, Wis., USA).

Primers of use in the present invention may be prepared by any number of conventional DNA synthesis methods. Primers described herein after were designed using the PrimerSelect program and manufactured by Invitrogen Corporation (Invitrogen NZ Limited, Auckland, New Zealand) in desalted form.

It will be appreciated that the usefulness of any primer of use in the invention may be evaluated, at least notionally, using appropriate software and the INHA DNA sequence information. Such software packages include, for example, PC Oligo5 (National Bioscience Inc) or Amplify (University of Wisconsin), and the PrimerSelect program previously mentioned herein.

Amplification is conducted according to conventional procedures in the art to which this invention relates; such as described in U.S. Pat. No 4,683,202. By way of exemplification PCRs according to the invention will generally include 0.1 μM-1 μM of each primer, 200 μM each dNTP, 3-7 mM MgCl₂, and 1U Taq DNA polymerase. Further, exemplary PCR cycling conditions include: denaturation at a temperature of 94° C. for 30 to 60 seconds, annealing at a temperature of from 45° C. to 62° C. for 30 to 60 seconds and extension at a temperature of 72° C. for 30 to 60 seconds. Preferably between 30 and 35 cycles are run. Further exemplary methodology is provided herein after under the heading “Examples”.

It will be appreciated by those of ordinary skill in the art that the PCR conditions provided herein are merely exemplary and may be varied so as to optimise conditions where, for example, alternative PCR cyclers or DNA polymerases are used, where the quality of the template DNA differs, or where variations of the primers not specifically exemplified herein are used, without departing from the scope of the present invention. The PCR conditions may be altered or optimised by changing the concentration of the various constituents within the reaction and/or changing the constituents of the reaction, altering the number of amplification cycles, the denaturation, annealing or extension times or temperatures, or the quantity of template DNA, for example. Those of skill in the art will appreciate there are a number of other ways in which PCR conditions may be optimised to overcome variability between reactions.

It will be understood that where not specifically exemplified herein appropriate PCR annealing temperatures for any primer within the scope of the present invention may be derived from the calculated melting temperature of that primer. Such melting temperatures may be calculated using standard formulas, such as that described in Sambrook and Russell, Molecular Cloning: A Laboratory Manual, 2001, Cold Spring Harbor Laboratory Press, Cold Spring Harbor, N.Y. As will be understood by those of ordinary skill in the art to which this invention relates annealing temperatures may be above or below the melting temperature but generally an annealing temperature of approximately 5° C. below the calculated melting temperature of the primer may be suitable.

Exemplary PCR conditions for specific primer pairs are provided herein after under the section entitled “Examples”.

Skilled persons will readily appreciate various cloning techniques which may be used to amplify specific target nucleic acid sequences for further analysis. Such techniques are standard in the art and are described for example in Sambrook et al (as herein before mentioned).

Nucleic acids can be analysed to confirm the nature of the genetic markers described herein according to any one of a number of known techniques. Appropriate techniques include for example polymerase chain reaction (PCR), including allele-specific PCR (Rano & Kidd, 1989), gel electrophoresis, Southern blotting, direct sequencing, restriction fragment length polymorphism (RFLP), single-strand confirmation polymphism (SSCP), (Orita et al, 1989), LCR (ligase chain reaction), denaturing gradient gel electrophoresis (DGGE), (Wartell et al, 1990; Sheffield et al, 1989), the use of allele-specific oligonucleotides (ASOs), (Conner et al, 1983), the use of proteins which recognize nucleic acid mismatches, such as E.coli mutS protein (Modrich, 1991), RNAse protection assays (Finkelstein et al, 1990; Kinsler et al, 1991), oligo array hybridisation, denaturing HPLC (dHPLC), fluorescence quenching PCR (TaqMan™, Applied Biosystems, CA 94404, USA), and matrix-assisted laser desorption/ionisation time-of-flight mass spectroscopy (MALDI-TOF MS). Combinations of two or more of such techniques may be used. Such combination may increase the sensitivity of the analysis being conducted.

The technique(s) used will depend on the nature of the marker to be detected as will be appreciated by skilled persons. For example, SNPs, such as −16C>T of INHA may be analysed using those techniques capable of resolving a single nucleotide difference between sequences; for example, direct sequencing or LCR, allele-specific PCR, RFLP, SSCP, DGGE, using allele-specific oligonucleotides (ASOs), or proteins which recognize nucleic acid mismatches, oligo array hybridisation, dBPLC, fluorescence quenching PCR and matrix MALDI-TOF MS.

Any one or more of the techniques mentioned hereinbefore (including for example, SSCP, RFLP, DGGE, dHPLC and direct sequencing) may be used to analyse the nature of the polymorphic repeat of the invention to identify the haplotype of a subject.

Persons of ordinary skill in the art to which the invention relates will appreciate how to implement the techniques above, especially having regard to the invention described herein, the nucleic acid sequence of the INHA gene, and the guidelines and instructions in any documents referenced herein which are incorporated herein by reference.

It should be appreciated that certain of the techniques of use in analysing or detecting the genetic markers of the invention will utilise one or more oligonucleotides which hybridise to a region of the INHA gene encompassing the marker, adjacent the marker or flanking the marker. Such oligonucleotides may be DNA or RNA and include the PCR primers referred to herein above as well as appropriate LCR primers and probes of use in RFLP analysis and the like.

Persons of ordinary skill in the art to which the invention relates will readily appreciate appropriate oligonucleotides of use in the invention having regard to the nucleic acid sequence of the INHA gene, the nature of the genetic markers to be analysed, and the general principles of nucleic acid hybridisation. Primers of use in PCR and LCR, for example, may be designed with the assistance of one or more of the computer programs hereinbefore described.

Oligonucleotide primers and probes may be made in accordance with chemical synthesis methods standard in the art. Alternatively they may be generated using standard recombinant techniques.

It will be appreciated that while oligonucleotide primers or probes of use in the methods of the invention may have 100% complementarity to their target region on the INHA gene, they may contain one or more non-complementary nucleotides at a particular position while still retaining their specificity in respect of the diagnostic assay being performed. It will be appreciated that in certain cases, the oligonucleotides will be designed such that a mismatch at a particular nucleotide position is indicative of the nature of the genetic marker being analysed and ultimately of the subjects predisposition to POF. By way of example, a mismatch in the nucleotide present at the 3′ end of an LCR primer will inhibit the reaction providing an indication of the nature of the nucleotide at that position on the INHA gene; in the case of the polymorphism −16C>T an LCR primer may be designed to have an A at the 3′ end, if the template DNA obtained from a subject has a C at the −16 position there will be a mismatch and the reaction will not occur indicating the subject does not carry the protective haplotype discussed elsewhere herein. Mismatches may similarly be utilised in techniques including RNAse protection assays and allele-specific PCR, as well as in fluorescence quenching PCR, for example. Oligonucleotide probes of use in the invention will generally hybridise to their target nucleic acid under stringent hybridisation conditions, as outlined in Sambrook et al (as herein before referenced).

As mentioned herein before, the oligonucleotide primers exemplified herein after under the heading “Examples” may be used in the amplification and analysis of the genetic markers of the invention. Having regard to the description provided herein, and the particular detection technique to be used, persons of skill in the art to which the invention relates will readily appreciate further appropriate primers of use in the invention.

In addition, nucleic acid probes of use in analysis and detection of the genetic markers of the invention are provided herein after under the heading “Examples”.

Oligonucleotide primers and probes used for detection and/or analysis of the genetic markers of the invention may be modified to facilitate such detection. Similarly, nucleic acid products obtained using techniques such as PCR may be modified to facilitate detection and/or analysis. For example, the nucleic acid molecules may be labelled to facilitate visual identification using techniques standard in the art. By way of example nucleic acids may be radio-labelled using P³² as may be described in Sambrook et al, Molecular Cloning (herein before detailed). Further, nucleic acids may be appropriately labelled for use in colorigenic, fluorogenic or chemiluminescence procedures.

Specific examples of techniques used to analyse the genetic markers of the invention to identify a subject's genotype for a particular marker or series of markers are provided herein after under the section entitled “Examples”. It will be appreciated that the methods of the invention may employ one or more control samples. Such control samples may be positive or negative controls for a particular genetic marker. The type of control samples used may vary depending on such factors as the nature of the genetic marker being analysed and the specific technique being used for such detection and analysis. Positive controls may include samples having known nucleic acid sequences. Negative controls may include samples having no nucleic acid present. By way of general example, in analysing the −16C>T SNP using LCR positive control samples could include nucleic acid known to have a C at position −16 and/or those samples known to include nucleic acid having a T at position −16. Further exemplary control samples of use in RFLP analysis are provided herein after under the heading “Examples”.

The inventors contemplate the methods of the invention including steps in addition to analysis of the genetic markers of the invention, or alternatively, analysis of the genetic markers of the invention being used as an adjunct to existing methodology used to detect predisposition to POF, diagnose POF or predict onset of infertility. For example, the analysis of the genetic markers could be combined with analysis of one or more additional factors or indicators of risk of, or protection from, infertility or POF, as well as those factors of use in diagnosing infertility or POF. Persons of skill in the art to which the invention relates will readily appreciate additional indicators whose analysis could be useful to this end (see for example, Bukman and Heineman (41)). However, by way of example, distinct genetic markers known to have a correlation with POF, including mutations in the Inhibin alpha gene (10) and FOXL2 gene (17), represent appropriate indicators. In addition, elevated levels of serum FSH (>40 IU/L for example) and lowered levels of serum inhibin and/or oestradiol are commonly used indicators of POF or impending menopause. A further example is low folicular reserve.

Persons of skill in the art to which the invention relates will readily appreciate means of analysing such additional indicators. Relevant indicators and tests are discussed for example in Bukman and Heineman, 2001 (41). By way of example, the following techniques may be of use: exogenous FSH ovarian reserve test; predicting ovarian reserve using endocrinology; ultrasound tests including antral follicle count, ovarian volume, ovarian stromal peak systolic velocity including waveform and pulsatility index, and ovarian follicular vascularity; measurement of blood levels of Follicle Stimulating Hormone (FSH), Luteinising Hormone (LH), FSH:LH ratios, oestradiol (E₂), and inhibin A and/or B using basal blood testing; ovarian bioposy; and, gonadotropin analogue stimulating test. A further example is the Clomiphene Challenge Test (CCT), which is a variation of baseline FSH measurement (approximately day 3), may provide an earlier warning sign of diminished ovarian reserve than baseline testing alone. The CCT involves determination of the day 3-5 FSH level, administration of clomiphene 100 mg per day on days 5-9 and re-testing of the FSH level on day 10. Finally, an ultrasound measurement of the antral follicles, or small follicles available to be stimulated may be used.

Combining the analysis of one or more of the genetic markers of the present invention with the analysis of one or more additional factors or indicators of relevance to infertility or POF may increase the power of the methods of the present invention in determining a subject's predisposition to POF. Such combinations are particuarly preferable where one wishes to diagnose POF or predict the onset of infertility.

As described herein before, the genetic markers in the IHNA gene in accordance with the invention have application in detecting whether a female subject has a predisposition or susceptibility to POF, in the diagnosis of POF and/or in predicting onset of infertility. This may allow for the early identification of susceptible individuals and thus allow implementation of approaches to help prevent or delay the onset of POF and infertility. Testing may enable subjects to make important life decisions (e.g. early child bearing).

### EXAMPLES

Materials and Methods

Patient Information and DNA Extraction

50 New Zealand and 20 Slovenian women, with POF, were recruited for this study by the Department of Obstetrics and Gynaecology in Auckland, New Zealand and the Department of Obstetrics and Gynaecology in Ljubljana, Slovenia. POF was defined as cessation of menses for a duration of six months or more before the age of 40 years, along with a FSH concentration of >40 IU/l. A complete medical and gynaecological history was taken from each patient as previously described (10). 50 normal control samples were obtained from the general population of New Zealand and 20 from the general population of Slovenia.

Genomic DNA was extracted from 10 ml samples of blood as previously described (10) and 100 ng was used as a template in a PCR.

Restriction Fragment Length Polymorphism Analysis (RFLP).

To rapidly screen all 70 patients and 70 controls for the −16C>T polymorphism an RFLP assay was performed as described previously (19).

DNA Sequencing of the INHA Promoter Region

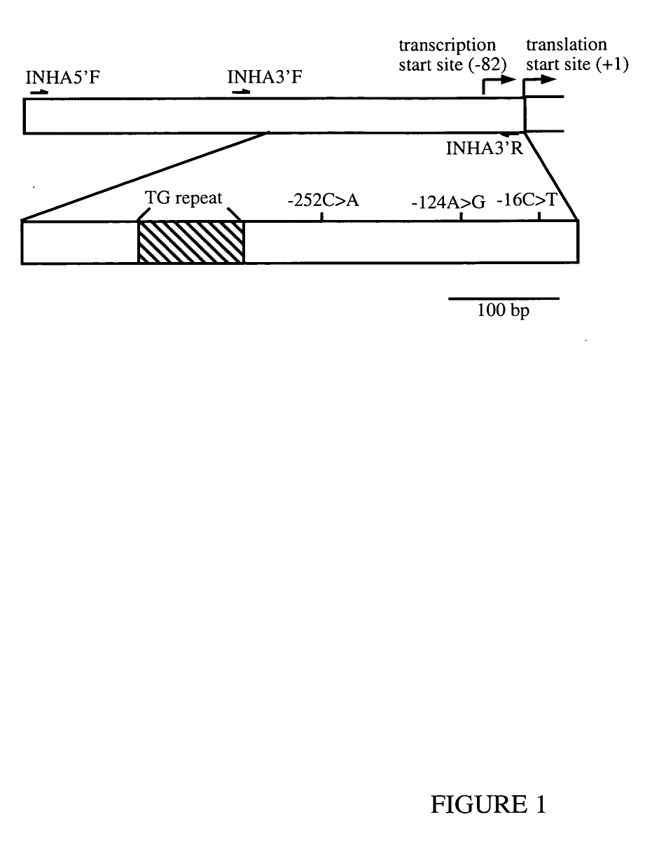

Primers were designed that flanked a 1 kb region of the INHA promoter (FIG. 1) using the Primer Select module in the DNAStar computer program from Lasergene 1994 (DNASTAR Inc., Madison, Wis., USA) and are as follows:

INHA5′F: 5′GCTCCCGGCTCGCCTCCTTACC3′
(nucleotides 42262-42284,
accession number AC009955)
INHA3′R: 5′CCTGGCCCTGCTAGTGGGGAACTC3′
(nucleotides 43257-43234,
accession number AC009955)

Reaction conditions were 0.4 μM of each primer, 0.2 mM of each dNTP, 0.625U Taq polymerase, 1×Q solution and 1×PCR buffer (Qiagen GmbH, Hilden, Germany) in a 25 μl total volume. 30 cycles of PCR were performed, consisting of 1 minute at 94° C., 1 minute at 62° C. and 2 minutes at 72° C., with a final 10 minute extension at 72° C. PCR products to be sequenced were purified using Roche's High Pure PCR Product

Purification Kit (Roche Diagnostics GmbH, Mannheim, Germany). Sequencing reactions were performed using the ABI PRISM™ BIG DYE Terminator Sequencing Kit under standard conditions with an annealing temperature of 55° C. Sequencing products were separated either on a ABI PRISM™ 377 DNA Sequencer XL machine or a ABI PRISM™ 3100 GeneticAnalyzer (PE Biosystems, Foster City, Calif., USA) at the Centre for Gene Technology, University of Auckland. Primers used for sequencing were INHA5′F and INHA3′R (FIG. 1).

Forced Restriction Fragment Length Polymorphism Analysis (FRFLP)

To confirm the sequencing data and to rapidly screen all New Zealand samples for the −124A>G polymorphism, identified in the INHA promoter, a FRFLP assay was devised. Primers were designed to amplify the region of INHA promoter containing the polymorphism, such that a Sau3A I restriction site was introduced by the reverse primer into samples containing an A at position −124, but not those containing a G. The primers were:

FRFLPF: 5′AGGTCGCTTGAGGCGAAATCCTTCC3′
FRFLPR: 5′TCCCACACCCACCCTCTTCTACCCTTCTGA3′

PCR conditions were as above with the exception that an extension time of 1 minute and no Q solution were used. A Sau3A I digestion resulted in the 196 bp PCR fragment forming two fragments of 168 bp and 28 bp in the presence of the A allele.

Single-Stranded Conformation Polymorphism (SSCP)

To rapidly screen all New Zealand samples for the polymorphic repeat identified in the INHA promoter, an SSCP assay was used. The region of the promoter containing the repeat was amplified by PCR using primers INHA3′F: 5′TATTGAAAGGGGCCCCAGAAGGTC3′ (nucleotides 42721-42744, accession number AC009955) and INHA3′R. PCR conditions used were the same as for the FRFLP assay. SSCP was performed as described previously (10), using a 14% (w/v) polyacrylamide gels without glycerol.

Results

50 New Zealand and 20 Slovenian POF patients, and the same number of controls, were screened for the −16C>T (FIG. 1) transition in the 5′UTR of INHA. 29 of 70 (41.4%) controls were heterozygous for this transition and 2 (2.9%) controls were homozygous for T. However, only 18 of 70 (25.7%) POF patients were heterozygous and no T homozygotes were identified. These results indicate that the T allele is significantly under-represented in the POF patient population (Fisher's exact test, 2-Tail: P=0.033).

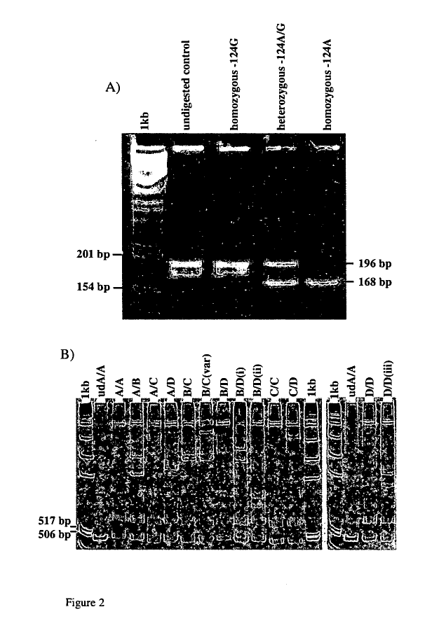

To analyse the INHA promoter sequence upstream of the −16C>T SNP, a ˜1 kb fragment of 5′UTR was amplified by PCR and sequenced in 38 of the New Zealand POF patients and 10 of the New Zealand controls. The imperfect TG repeat element located ˜300 bp upstream of the ATG start site (FIG. 1) was found to be highly polymorphic in both populations. To determine the exact sequences of both alleles from several of the samples, it was necessary to subclone them into pCR®4Blunt-TOPO® and then sequence using vector primers. Initially, 5 haplotypes were identified [A-D and D(i), table I]. A −124A>G SNP was also identified and shown to be inherited co-ordinately with the polymorphic repeat, with haplotypes A and B having −124A and haplotypes C, D and D(i), −124G. No other variants were identified. To rapidly type the remaining New Zealand samples, a FRFLP/SSCP combined assay was devised (FIG. 2) and all 50 New Zealand POF patients and 50 controls were screened by this method. Where a novel or unclear SSCP pattern was identified, the sample was sequenced and where necessary cloned into pCR®4Blunt-TOPO® prior to sequencing. In total, 7 repeat polymorphism haplotypes were typed (A-D and D(i-iii), table I) along with a third SNP, −252C>A (FIG. 1) in one of the control samples (genotype B/C). Haplotype D(i) was found in a single POF patient and haplotypes D(ii) and D(iii) were each identified in single controls. The repeat region varied in length from 94 nucleotides in haplotype A to only 76 nucleotides in haplotype C. Table II details the genotypes identified in both the POF and control populations. The results indicate that in the New Zealand population, repeat polymorphism haplotype C is in linkage disequilibrium with −16T, as every sample that was heterozygous for −16T was also heterozygous for haplotype C and the two controls that were homozygous for one were also homozygous for the other. Interestingly, haplotype C contains the shortest repeat polymorphism, being at least 10 nucleotides shorter than the other variants. The C haplotype was underrepresented in the patients (14%), compared to controls (23%) (Table III).

TABLE I
Repeat polymorphisms in the INHA promoter

| | | Length of | | |
|---|---|---|---|---|
| | | repeat | | |
| Haplotype | Repeat sequence˜−300 bp | region | −124 bp | −16 bp |
| A | (TG)₈(AG)₅(TG)₆AGAATT(TG)₆AG(TG)₅AG(TG)₅AG(TG)₆ | 94 | A | C |
| B | (TG)₈(AG)₃(TG)₆AGAATT(TG)₆AG(TG)₄AG(TG)₅AG(TG)₆ | 88 | A | C |
| C | (TG)₉(AG)₃(TG)₅AGAATT(TG)₆AG(TG)₅AG(TG)₅ | 76 | G | T |
| D | (TG)₉(AG)₄(TG)₆AGAATT(TG)₆AG(TG)₅AG(TG)₁₀ | 90 | G | C |
| D(i) | (TG)₈(AG)₄(TG)₆(AG)₄(TG)₅AG(TG)₅AG(TG)₁₀ | 88 | G | C |
| D(ii) | (TG)₉(AG)₄(TG)₆AGAATT(TG)₆AG(TG)₅AG(TG)₉ | 88 | G | C |
| D(iii) | (TG)₉(AG)₃(TG)₅AGAATT(TG)₆AG(TG)₅AG(TG)₁₀ | 86 | G | C |

TABLE II
INHA promoter genotypes in POF patients and controls

| Genotype | Controls | POF patients |
|---|---|---|
| A/A | 13 | 13 |
| A/B | 5 | 4 |
| A/C | 11 | 10 |
| A/D | 7 | 12 |
| B/C | 4 | 3 |
| B/D | 1 | 3 |
| B/D(i) | 0 | 1 |
| B/D(ii) | 1 | 0 |
| C/C | 1 | 0 |
| C/D | 6 | 1 |
| D/D | 0 | 3 |
| D/D(iii) | 1 | 0 |
| Total | 50 | 50 |

TABLE III
INHA promoter genotypes according to allele in
POF patients and controls

| Genotype | Controls | POF patients |
|---|---|---|
| A | 49 | 52 |
| B | 11 | 11 |
| C | 23 | 14 |
| D | 17 | 23 |
| Total | 100 | 100 |

Discussion

Previous studies have revealed the importance of inhibin in follicular development and have shown a strong association between a coding variant, 769G>A, in INHA, that causes a non-conservative amino acid change, A257Thr, and POF. More recent studies have identified a pair of linked INHA SNP's, 129C>T and 675C>T (19). The 129C>T SNP has been associated with susceptibility to POF (18).

The inventor(s) have screened 70 POF patients and 70 controls from the populations of New Zealand and Slovenia and identified a −16C>T SNP and detected the T allele in 31 of 70 (44.3%) of the control population, but only 18 of 70 (25.7%) of POF patients, showing a statistically significant difference between the two populations (Fisher's exact test, 2-Tail: P=0.033). Interestingly, it is the less common variant (−16T) that is under-represented in the POF patients when compared with the general population. The inventors believe that the −16T allele represents a susceptibility allele, and protects women from the early development of POF. An alternative presentation of this data is that the −16C allele predisposes women to an early menopause.

Despite the association between the −16C>T SNP and POF, it is unlikely that a single nucleotide polymorphism located between the transcription and translation start sites, could on its own significantly affect expression from INHA. So, the inventor(s) sequenced a ˜1 kb region of the promoter upstream from this SNP, in 38 New Zealand POF patients and 10 controls. The region of promoter sequenced contained several known response elements and had previously been shown to have promoter activity in both human and murine reporter gene assays (22, 23). From this initial study the inventors discovered an extended imperfect TG repeat at ˜−300 which is polymorphic in both the POF patient and control populations, with the repeat region ranging from 76 to 94 bp in length, and that −16T is linked to the shortest haplotype (haplotype C). Analysis of the remaining New Zealand samples by FRFLP and SSCP confirmed this linkage and lead to the discovery of a total of seven different variants at the repeat locus.

Since −16T is under-represented in the 70 New Zealand and Slovenian POF patients and is linked to repeat polymorphism haplotype C in the 50 New Zealand POF patients and 50 New Zealand controls, the inventors believe that the TG repeat polymorphism haplotype C is also under-represented in the POF patient population. The haplotype C was underrepresented in patients compared to controls (Table III). TG repeats are widely dispersed throughout eukaryotic genomes, with a copy number of ˜10⁵ in the human genome (31, 32).

Whilst not wishing to be bound by any particular theory, the inventors propose that in vivo, INHA alleles with haplotype C express a greater amount of INHA than those with other haplotypes, which leads to an increase in inhibin levels and thus a decrease in FSH levels, which in turn leads to a decrease in the rate of follicle loss, and thus a later menopause.

The invention has been described herein with reference to certain preferred embodiments, in order to enable the reader to practice the invention without undue experimentation. However, a person having general skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country.

Throughout this specification, and any claims which follow, unless the context requires otherwise, the words “comprise”, “comprising” and the like, are to be construed in an inclusive sense as opposed to an exclusive sense, that is to say, in the sense of “including, but not limited to”.

### REFERENCES

1. Ling N, Ying S Y, Ueno N, Esch F, Denoroy L, Guillemin R 1985 Isolation and partial characterization of a Mr 32,000 protein with inhibin activity from porcine follicular fluid. Proc Natl Acad Sci USA 82:7217-21.
2. Miyamoto K, Hasegawa Y, Fukuda M, Nomura M, Igarashi M, Kangawa K, Matsuo H 1985 Isolation of porcine follicular fluid inhibin of 32K daltons. Biochem Biophys Res Commun 129:396-403.
3. Rivier J, Spiess J, McClintock R, Vaughan J, Vale W 1985 Purification and partial characterization of inhibin from porcine follicular fluid. Biochem Biophys Res Commun 133:120-7.
4. Robertson D M, Foulds L M, Leversha L, Morgan F J, Hearn M T, Burger H G, Wettenhall R E, de Kretser D M 1985 Isolation of inhibin from bovine follicular fluid. Biochem Biophys Res Commun 126:220-6.
5. Vale W, Rivier C, Hsueh A, Campen C, Meunier H, Bicsak T, Vaughan J, Corrigan A, Bardin W, Sawchenko P, et al. 1988 Chemical and biological characterization of the inhibin family of protein hormones. Recent Prog Horm Res 44:1-34
6. McLachlan R I, Robertson D M, Healy D L, Burger H G, de Kretser D M 1987 Circulating immunoreactive inhibin levels during the normal human menstrual cycle. J Clin Endocrinol Metab 65:954-61.
7. Reddi K, Wickings E J, McNeilly A S, Baird D T, Hillier S G 1990 Circulating bioactive follicle stimulating hormone and immunoreactive inhibin levels during the normal human menstrual cycle. Clin Endocrinol (Oxf) 33:547-57.
8. Ling N, Ying S Y, Ueno N, Shimasaki S, Esch F, Hotta M, Guillemin R 1986 A homodimer of the beta-subunits of inhibin A stimulates the secretion of pituitary follicle stimulating hormone. Biochem Biophys Res Commun 138:1129-37.
9. Vale W, Rivier J, Vaughan J, McClintock R, Corrigan A, Woo W, Karr D, Spiess J 1986 Purification and characterization of an FSH releasing protein from porcine ovarian follicular fluid. Nature 321:776-9.
10. Shelling A N, Burton K A, Chand A L, van Ee C C, France J T, Farquhar C M, Milsom S R, Love D R, Gersak K, Aittomaki K, Winship I M 2000 Inhibin: a candidate gene for premature ovarian failure. Hum Reprod 15:2644-9.
11. Coulam C B, Adamson S C, Annegers J F 1986 Incidence of premature ovarian failure. Obstet Gynecol 67:604-6.
12. Shelling A N 2000 X chromosome defects and premature ovarian failure. Aust N Z J Med 30:5-7.
13. Aittomaki K, Lucena J L, Pakarinen P, Sistonen P, Tapanainen J, Gromoll J, Kaskikari R, Sankila E M, Lehvaslaiho H, Engel A R, et al. 1995 Mutation in the follicle-stimulating hormone receptor gene causes hereditary hypergonadotropic ovarian failure. Cell 82:959-68.
14. Beau I, Touraine P, Meduri G, Gougeon A, Desroches A, Matuchansky C, Milgrom E, Kuttenn F, Misrahi M 1998 A novel phenotype related to partial loss of function mutations of the follicle stimulating hormone receptor. J Clin Invest 102:1352-9.
15. Touraine P, Beau I, Gougeon A, Meduri G, Desroches A, Pichard C, Detoeuf M, Paniel B, Prieur M, Zorn J R, Milgrom E, Kuttenn F, Misrahi M 1999 New natural inactivating mutations of the follicle-stimulating hormone receptor: correlations between receptor function and phenotype. Mol Endocrinol 13:1844-54.
16. Latronico A C, Anasti J, Arnhold I J, Rapaport R, Mendonca B B, Bloise W, Castro M, Tsigos C, Chrousos G P 1996 Brief report: testicular and ovarian resistance to luteinizing hormone caused by inactivating mutations of the luteinizing hormone-receptor gene. N Engl J Med 334:507-12.
17. Harris S E, Chand A L, Winship I M, Gersak K, Aittomaki K, Shelling A N 2002 Identification of novel mutations in FOXL2 associated with premature ovarian failure. Mol Hum Reprod 8:729-33.
18. Marozzi A, Porta C, Vegetti W, Crosignani P G, Tibiletti M G, Dalpra L, Ginelli E 2002 Mutation analysis of the inhibin alpha gene in a cohort of Italian women affected by ovarian failure. Hum Reprod 17:1741-5.
19. Montgomery G W, Duffy D L, Hall J, Haddon B R, Kudo M, McGee E A, Palmer J S, Hsueh A J, Boomsma D I, Martin N G 2000 Dizygotic twinning is not linked to variation at the alpha-inhibin locus on human chromosome 2. J Clin Endocrinol Metab 85:3391-5.
20. Meunier H, Rivier C, Evans R M, Vale W 1988 Gonadal and extragonadal expression of inhibin alpha, beta A, and beta B subunits in various tissues predicts diverse functions. Proc Natl Acad Sci USA 85:247-51.
21. Pei L, Dodson R, Schoderbek W E, Maurer R A, Mayo K E 1991 Regulation of the alpha inhibin gene by cyclic adenosine 3′,5′-monophosphate after transfection into rat granulosa cells. Mol Endocrinol 5:521-34.
22. Debieve F, Thomas K 2002 Control of the human inhibin alpha chain promoter in cytotrophoblast cells differentiating into syncytium. Mol Hum Reprod 8:262-70.
23. Su J G, Hsueh A J 1992 Characterization of mouse inhibin alpha gene and its promoter. Biochem Biophys Res Commun 186:293-300.
24. Ardekani A M, Romanelli J C, Mayo K E 1998 Structure of the rat inhibin and activin betaA-subunit gene and regulation in an ovarian granulosa cell line. Endocrinology 139:3271-9.
25. Yoshida E, Tanimoto K, Murakami K, Fukamizu A 1998 Isolation and characterization of 5′-regulatory region of mouse activin beta A subunit gene. Biochem Mol Biol Int 44:325-32.
26. Tanimoto K, Yoshida E, Mita S, Nibu Y, Murakami K, Fukamizu A 1996 Human activin betaA gene. Identification of novel 5′ exon, functional promoter, and enhancers. J Biol Chem 271:32760-9.
27. Feng Z M, Li Y P, Chen C L 1989 Analysis of the 5′-flanking regions of rat inhibin alpha- and beta-B-subunit genes suggests two different regulatory mechanisms. Mol Endocrinol 3:1914-25.
28. Dykema J C, Mayo K E 1994 Two messenger ribonucleic acids encoding the common beta B-chain of inhibin and activin have distinct 5′-initiation sites and are differentially regulated in rat granulosa cells. Endocrinology 135:702-11.
29. Feng Z M, Wu A Z, Chen C L 1995 Characterization and regulation of two testicular inhibin/activin beta B-subunit messenger ribonucleic acids that are transcribed from alternate initiation sites. Endocrinology 136:947-55.
30. Nishi Y, Yanase T, Mu Y, Oba K, Ichino I, Saito M, Nomura M, Mukasa C, Okabe T, Goto K, Takayanagi R, Kashimura Y, Haji M, Nawata H 2001 Establishment and characterization of a steroidogenic human granulosa-like tumor cell line, KGN, that expresses functional follicle-stimulating hormone receptor. Endocrinology 142:437-45.
31. Hamada H, Petrino M G, Kakunaga T 1982 A novel repeated element with Z-DNA-forming potential is widely found in evolutionarily diverse eukaryotic genomes. Proc Natl Acad Sci USA 79:6465-9.
32. Hamada H, Kakunaga T 1982 Potential Z-DNA forming sequences are highly dispersed in the human genome. Nature 298:396-8.
33. Dobi A, v Agoston D 1998 Submillimolar levels of calcium regulates DNA structure at the dinucleotide repeat (TG/AC)n. Proc Natl Acad Sci USA 95:5981-6.
34. Keelan J, Song Y, France J T 1994 Comparative regulation of inhibin, activin and human chorionic gonadotropin production by placental trophoblast cells in culture. Placenta 15:803-18.
35. Hsu S Y, Lai R J, Nanuel D, Hsueh A J 1995 Different 5′-flanking regions of the inhibin-alpha gene target transgenes to the gonad and adrenal in an age-dependent manner in transgenic mice. Endocrinology 136:5577-86.
36. WO 00/50638
37. Finkelstein, J., et al. (1990). Genomics 7:167-172.
38. Kinszler, K. W., et al., (1991). Science 251:1366-1370.
39. Antonarakis and the Nomenclature Working Group (1998). Human Mutation 11:1-3
40. den Dunnen J T, Antonarakis S E. 2000. Mutation nomenclature extensions and suggestions to describe complex mutations: a discussion. Human Mutation. 15:7-12.
41. Buckman and Heineman. 2001. Ovarian reserve testing and the use of prognostic models in patients with subfertility. Human Reproduction Update, Vol. 7, No. 6, pp. 581-590

1. A method to detect whether a female subject is predisposed to POF, the method comprising analysing at least one or more polymorphism in the INHA gene chosen from the group consisting:
−1 24A>G;
−16C>T;
TG repeat (as herein after described); and
One or more polymorphism in the INHA gene which is in linkage disequilibrium with one or more of −124A>G, −16C>T or TG repeat (as herein after described).

2. A method for diagnosing POF in a female subject, the method comprising analysing at least one or more polymorphism in the INHA gene chosen from the group consisting:
−124A>G;
−16C>T;
TG repeat (as herein after described); and,
One or more polymorphism in the INHA gene which is in linkage disequilibrium with one ore more of −124A>G, −16C>T or TG repeat (as herein after described).

3. A method for predicting the onset of infertility in a female subject, the method comprising at least the analysis of one or more polymorphism in the INHA gene chosen from the group consisting:
−124A>G;
−16C>T;
TG repeat (as herein after described); and
One or more polymorphism in the INHA gene which is in linkage disequilibrium with one or more of −124A>G, −16C>T or TG repeat (as herein after described).

4. A method as claimed in claim 1 wherein the one or more polymorphism in the INHA gene which is in linkage disequilibrium is 531C>T.

5. A method to detect whether a female subject is predisposed to POF, the method comprising determining the subjects genotype in respect of a TG repeat polymorphism (as herein after described) in the INHA gene by analysing one or more of the polymorphisms chosen from the group consisting:
−16C>T; and,
−124A>G.

6. A method for diagnosing POF in a female subject, the method comprising determining the subjects genotype in respect of a TG repeat polymorphism (as herein after described) in the INHA gene by analysing one or more of the polymorphisms chosen from the group consisting:
−16C>T; and,
−124A>G.

7. A method for predicting the onset of infertility in a female subject, the method comprising determining the subjects genotype in respect of a TG repeat polymorphism (as herein after described) in the INHA gene by analysing one or more of the polymorphisms chosen from the group consisting:
−16C>T; and,
−124A>G.

8. A method as claimed in claim 1 wherein heterozygosity or homozygosity for haplotype C (as herein after described) of the TG repeat is indicative of protection against POF.

9. A method as claimed in claim 1 wherein heterozygosity or homozygosity for −16T (the T allele) is indicative of protection against POF.

10. A method as claimed in claim 1 wherein homozygosity for −124A is indicative of susceptibility or predisposition to POF, and predictive of potential onset of infertility.

11. A method as claimed in claim 1 wherein homozygosity or heterozygosity for −124G is indicative of protection against POF.

12. A method as claimed in claim 1 wherein analysis of one or more polymorphisms occurs via analysis of DNA encoding INHA.

13. A method as claimed in claim 1 wherein where a polymoprhism in linkage disequillibrium is analysed and said polymorphism is located in a coding region, the method involves analysis of RNA encoding INHA.

14. A method as claimed in claim 12 or 13 wherein analysis of one or more polymorphisms occurs using one or more of:
polymerase chain reaction (PCR);
gel electrophoresis;
Southern blotting;
Nucleic acid sequencing;
restriction fragment length polymorphism (RFLP);
single-strand confirmation polymphism (SSCP);
LCR (ligase chain reaction);
denaturing gradient gel electrophoresis (DGGE);
allele-specific oligonucleotides (ASOs);
proteins which recognize nucleic acid mismatches;
RNAse protection;
oligo array hybridisation;
denaturing HPLC (dHPLC); and, matrix-assisted laser desorption/ionization time-of-flight mass spectroscopy (MALDI-TOF MS).
